(19) United States
(12) Patent Application Publication
(10) Pub. No.: US 2023/0226369 A1
(43) Pub. Date: Jul. 20, 2023

(54) USE OF BLUE LIGHT FOR INDUCTION OF ANGIOGENESIS

(71) Applicants: URGO RECHERCHE INNOVATION DEVELOPPEMENT, Chenôve (FR); ANGIO GBR, Mannheim (DE)

(72) Inventors: Norbert GRETZ, Mannheim (DE); Marielle BOUSCHBACHER, Chambolle-Musigny (FR); Kejia KAN, Mannheim (DE); Mickael KEESE, Weinheim (DE); Prama PALLAVI, Mannheim (DE)

(21) Appl. No.: 18/010,266

(22) PCT Filed: Jun. 23, 2021
(86) PCT No.: PCT/IB2021/055562

§ 371 Date: Dec. 14, 2022

Related U.S. Application Data
(60) Provisional application No. 63/042,911, filed on Jun. 23, 2020.

(51) Int. Cl. A61N 5/06

(52) U.S. Cl. CPC A61N 5/0616; A61N 2005/0663

(57) ABSTRACT

A method of using blue light for inducing angiogenesis for treatment of wounds, burns, and other injuries, or to treat ischemic conditions, is provided. The method comprises irradiating a target with a visible light, wherein the visible light has a dominant emission wavelength ranging from 435 nm to 520 nm, and effective fluence of the visible light ranges from about 0.15 J/cm2 to about 17 J/cm².

### PRIORITY

This application claims the priority and benefit of U.S. Provisional Pat. Application No. 63/042,911, filed Jun. 23, 2020, the disclosure of which is hereby incorporated by reference in its entirety.

### BACKGROUND

Healing of a wound is a natural physiopathological process, by which the human and animal tissues are able to repair the lesions by specific processes of reparation and regeneration. Natural healing of a wound proceeds mainly according to three major chronological processes - the inflammatory phase, the proliferative phase (which includes the phases of granulation and epithelialization), and the phase of remodeling. See, e.g., Gonzalez et al., Wound healing - a literature review, Anais brasileiros de dermatologia vol. 91:5 (2016): 614-620. Each of these processes is characterized by specific cellular activities and is controlled by a multiplicity of signals of regulation (as well as positive and negative) which, collectively, orchestrate and frame the progression of the process of repair.

The first, inflammatory phase begins when blood vessels are ruptured, which is the event that starts formation of a clot (coagulation of blood) mainly made up of fibrin and fibronectin, and which will constitute a provisional matrix. This matrix fills the lesion partly and allows the migration, within the injured zone, of inflammatory cells recruited to ensure the debridement of the wound. This phase is characterized by the infiltration on the site of the lesion of many inflammatory cells (polynuclear cells, macrophages, etc.), ensuring the defense of the organism against possible foreign micro-organisms as well as the cleaning of the wound or debridement.

The second, proliferative phase includes two steps. The first step corresponds to the development of the granulation tissue, while the second step is epithelialization. Initially, a colonization of the wound occurs by migration and proliferation of fibroblasts. Then, the migration of endothelial cells, starting from the healthy vessels, allows neovascularization, or angiogenesis, of the injured tissue. Angiogenesis is the formation of new blood vessels from the preexisting vasculature. Endothelial cells multiply and then migrate from the peripheral healthy vessels, firstly in the form of cord-like hollow structures which then grow and thus lead to reconstitution of new vessels. Endothelial cells play a critical role in angiogenesis by repairing damaged or diseased tissues via the growth of blood vessels from existing vasculature. The basic steps of angiogenesis include endothelial cell proliferation, migration, and tubulogenesis (tube formation). Successful angiogenesis forms the basis of a tissue filler survival in the wound bed, and this in turn determines the outcome of the healing process during the granulation phase. In adults, endothelial cells are mostly quiescent, but angiogenesis may however be induced by angiogenic factors, such as the vascular endothelial growth factor (VEGF), when tissues become hypoxic, as occurs in pathologies such as, for example, wounds and cancers.

In the granulation tissue formed during wound healing, fibroblasts are activated and then differentiate into myofibroblasts that present important contractile properties. Actin microfilaments play an important role in reestablishment of endothelial vessel integrity and thus allow a contraction of the wound. The myofibroblasts play a main function in the formation and the contraction of granulation tissue, which leads to the healing of the lesion. The epithelialization step migration involves keratinocytes migration, starting from the edges of the wound, leading to the rebuilding of the new epidermis. At the final stage of a lesion’s healing, remodeling stage, reorganization, degradation, and resynthesis of the extracellular matrix take place.

Certain types of wounds do not heal correctly, for example, when there are abnormalities in the processes of the three key stages of the wound healing process. Certain wounds with a propensity to not heal properly and timely include chronic wounds such as, e.g., diabetic ulcer, venous or arterial ulcer (including venous leg ulcer), pressure ulcer or sores, second and third degree burns, skin grafts, amputation wounds, among others. Chronic wounds can be defined as wounds that do not exhibit signs of healing after six weeks from the appearance of the wound, regardless of the treatment applied. The speed and quality of the healing of a wound depend on intrinsic and extrinsic factors. This process of repair can thus be abnormally prolonged based on etiology of the wound, its state and its localization, occurrence of an infection caused by the presence of certain infectious agents (e.g., Staphylococcus aureus or Pseudomonas aeruginosa), preexisting pathology (e.g., diabetes, an immune deficiency, a venous insufficiency, etc), external environmental factors, or genetic factors affecting the wound healing process.

Further, wound healing can be complicated by ischemia and/or hypoxia that may occur in post-traumatic as well as in post-surgical conditions. The triad of ischemia, neuropathy and infection presents a challenge in treatment of foot ulcer and other lesions affecting diabetic patients.

Improved or alternative methods for wound treatment, including for chronic wounds, are needed.

### SUMMARY

Accordingly, in various aspects, the present invention relates to use of a visible light in the wavelength range of 435 to 520 nanometers (nm), in combination with specific effective fluence to promote or induce proliferation, migration and tube formation by endothelial cells. The visible light is applied to a target, such as an area of a subject’s body, to cause a therapeutic effect.

The inventors have surprisingly discovered that the blue light (e.g., visible light having a wavelength of 435 to 520 nm) having the effective fluence of about 17 J/cm2 or less promotes or induces angiogenesis. In some embodiments, effective fluence of the blue light can range from about 0.15 J/cm² to about 17 J/cm². In some embodiments, the effective fluence is from about 0.15 J/cm² to about 15 J/cm². In some embodiments, the effective fluence is from about 5 J/cm² to about 15 J/cm². In a preferred embodiment, the effective fluence is about 7.2 J/cm².

In some embodiments of the present disclosure, the incident power density of about 20 mW/cm² or less promotes or induces angiogenesis. In some embodiments of the present disclosure, in order to induce angiogenesis, the incident power density of blue light ranges from about 0.5 mW/cm² to about 20 mW/cm², or from about 1 mW/cm² to about 15 mW/cm², or from about 5 mW/cm² to about 15 mW/cm², or from about 7 mW/cm² to about 15 mW/cm². In some embodiments, the incident power density of blue light is about 10 mW/cm².

In some embodiments of the present disclosure, the dominant emission wavelength ranges from 435 to 520 nm, particularly from 450 to 490 nm, and more particularly from 450 to 460 nm. In an exemplary embodiment, the dominant emission wavelength is about 453 nm.

In some embodiments of the present disclosure, an irradiation time period is from about 5 minutes to about 15 minutes, particularly from about 7 minutes to about 15 minutes. In some embodiments, an irradiation time period is from about 10 minutes to about 15 minutes. For example, the irradiation time period can be about 12 minutes.

In some embodiments, a distance between a target and a source of blue light is between about 1 cm and about 15 cm, or between about 1 cm and about 10 cm, or between about 2 cm and about 7 cm, or about 5 cm.

In some embodiments, the incident power density of blue light is about 10 mW/cm², the irradiation time period is about 12 minutes, and a distance between a target is about 5 cm.

In some embodiments, the irradiation is applied at least during the granulation phase. In some embodiments, the irradiation is also applied during at least a portion of the inflammatory phase and/or the remodeling phase. However, in some embodiments, the irradiation is not applied during the inflammatory phase. In some embodiments, the irradiation is not applied during the remodeling phase.

The irradiation can be applied in various regimens. In some embodiments, the irradiation is provided in a regimen comprising irradiation from about 1 to 5 times per day, to about 1 to about 7 times per week. In some embodiments, the regimen is provided at least during (e.g., throughout) the granulation phase.

In some embodiments of the present disclosure, the target is a region of tissue in need of an induction of angiogenesis, which can be, for example, tissue affected by a wound (acute or chronic). In some embodiments, the tissue is damaged or the skin is wounded, such as a partial thickness or full thickness wound. The present disclosure can be used in treatments of wounds or surgical incisions, dermatological disorders involving impaired skin, dermatologic rare diseases involving impaired skin tissue, and treatments for use in medical dermatology and aesthetic medicine. In some embodiments, the methods of the present disclosure can be used to facilitate healing of skin grafts (e.g., autografts, allografts, or heterografts) or tissue surrounding prosthetic implants. In some embodiments the present disclosure can be used to treat vascular disorders, such as systemic scleroderma or atheriosclerotic lesions.

In some embodiments of the present disclosure, the blue light irradiation is administered directly on the target tissue. In some embodiments, the blue light irradiation passes through a product adapted to be in contact with the target tissue. For example, in some embodiments, the product that can be in contact with the target tissue can be a dressing, a strip, a bandage, a compression element, a Band-Aid©, a patch, a bag, a pouch, a strip, a gel, a film, a film-forming composition, or other product or combination thereof. In some embodiments, the product is a rigid or flexible support, such as, e.g., a dressing. In some embodiments, the dressing comprises at least a hydrocolloid or an adhesive layer that is used to attach the dressing to intact skin proximate a wound or damaged tissue that is the target for treatment. In some embodiments, the product through which blue light irradiation passes is translucent or it can be transparent.

### BRIEF DESCRIPTION OF THE DRAWINGS

FIG. 1 a schematic representation showing the endothelial cells proliferation, 24h hours after different exposure times with blue light irradiation (incident power density of 10 mW/cm² and 20 mW/cm²).

FIG. 2 is a schematic representation showing the endothelial cell apoptosis after irradiation with blue light at different incident power densities (10 mW/cm², 20 mW/cm² and 40 mW/cm²).

FIG. 3A is a schematic representation showing the endothelial cells proliferation, 24 hours after irradiation with blue light at different incident power densities.

FIG. 3B is a schematic representation showing the endothelial cells proliferation, 24 hours after irradiation with blue light at different effective fluences.

FIG. 4A is a schematic representation showing the endothelial cells migration, different times after irradiation with blue light at three incident power densities (10 mW/cm², 20 mW/cm², and 40 mW/cm²).

FIG. 4B is a schematic representation showing the endothelial cells migration, different times after irradiation with blue light at three effective fluences (7.2 J/cm², 14.4 J/cm², and 28.8 J/cm²).

FIG. 5 is a schematic representation showing the endothelial cells angiogenesis by the tube formation process (tube length in pixels (pc)), 6 hours after irradiation with blue light at three incident power densities (10 mW/cm², 20 mW/cm², and 40 mW/cm²).

FIG. 6A is an image illustrating results of Spheroid Sprouting Assay using control, non-irradiated cells.

FIG. 6B is an image illustrating results of Spheroid Sprouting Assay using cells irradiated with blue light at 10 mW/cm² power density.

FIG. 6C is an image illustrating results of Spheroid Sprouting Assay using cells irradiated with blue light at 40 mW/cm² power density.

### DETAILED DESCRIPTION

The present invention provides methods for irradiating target tissue in need of an induction or promotion of angiogenesis. The invention in various aspects and embodiments comprises irradiating target tissue with blue light under specific conditions to modulate activity of endothelial cells, in particular, to promote or induce angiogenesis through increasing cell viability, proliferation, migration, and/or tubulogenesis. The inventors have surprisingly discovered that certain parameters used for blue light irradiation provide the unexpected technical effect of promoting or inducing angiogenesis.

Accordingly, in some aspects of the present disclosure, a method for inducing angiogenesis is provided that comprises irradiating a target tissue with visible light, wherein the visible light has a dominant emission wavelength ranging from 435 nm to 520 nm, and wherein effective fluence ranges from about 0.15 J/cm² to about 17 J/cm².

In some embodiments, effective fluence of the blue light can range from about 0.15 J/cm² to about 17 J/cm². In some embodiments, the effective fluence is from about 5 J/cm² to about 15 J/cm². In some embodiments, the effective fluence is about 7.2 J/cm². In some embodiments, the incident power density ranges from about 8 mW/cm² to about 12 mW/cm². In some embodiments, the power density is about 10 mW/cm².

The methods in accordance with the present disclosure can be used for application to a tissue that is in need of repair, and in particular in need of angiogenesis to support wound healing. Embodiments of the present invention provide ways to induce angiogenesis in an effective and safe, and non-pharmacological manner. For example, the methods can be applied for treatment of a wound (acute or chronic). The wound can be an acute wound or it can be a chronic wound such as, e.g., venous ulcer, pressure ulcer or bedsore, amputation wound, diabetic ulcer, or other diabetes-related wound, which are often slow or difficult to heal. In order to enhance the process of wound healing, for both wounds which heal naturally and chronic wounds (i.e., those that do not heal after six week from their appearance, even if treated), the goal is to speed up the healing process at one or more of the inflammatory phase, proliferative phase (includes the granulation and epithelialization phases), and the remodeling phase of the tissue healing. For example, the method as described herein can be employed at least during the proliferative phase of wound healing. In some embodiments, the method as described herein is also employed during at least a portion of the inflammatory phase and/or remodeling phase. For example, the method can be employed during the proliferative phase and remodeling phases. In some embodiments, the method is employed during all phases of wound healing.

Phototherapy has been used to enhance certain steps in the process of wound healing. For example, photodynamic therapy and photobiomodulation have been described. Photodynamic therapy is a method that uses a photosensitizer, or photosensitizing agent, which is an agent disposed or injected near skin or wound cells and activated by a light of a specific wavelength. Photosensitizers have the ability to interact with the nearby skin cells when exposed to a light with a specific wavelength. Photodynamic therapy is thus an indirect phototherapy, because the light is provided to the photosensitizer to treat the skin cells, not directly to the skin cells.

Photobiomodulation is a method that allows providing a biological effect on skin or wound cells directly, i.e. without the need of any product or composition to transpose or potentiate the biological effect engendered by the light source. This method can be distinguished from the photodynamic therapy that requires the intervention of an intermediate product (photosensitizer or a photosensitizing agent) between the light source and cells, to potentiate the biological effect of the light on the cells. In other words, in photobiomodulation, light has a direct effect on cells, whereas, in photodynamic therapy, light has an indirect effect on cells via the activated photosensitizer.

Aspects of the present disclosure pertain to photobiomodulation that makes use of blue light. Blue light is a color in the visible spectrum, with the wavelength of from 380 nm and 500 nm.

The use of blue light has been described. For example, U.S. Pat. Application Publication 2019/0175936, which is hereby incorporated by reference in its entirety, is directed to a photobiomodulation light source device that can provide blue light to promote or induce growth and proliferation of keratinocytes and fibroblasts. The light source can be, e.g., a light-emitting diode (LED) source, a laser light source, or another suitable source. As another example of photobiomodulation, International Application No. PCT/EP2019/05192, which is incorporated by reference herein in its entirety, describes a light source device that can reduce contaminating and/or pathogenic agents’ growth and number. It was also shown that stimulation with both green- and red-pulsed LED light significantly increases Human Umbilical Vein Endothelial Cells (HUVEC) proliferation and migration, while treatment with blue light was described to be ineffective. Rohringer et al., The impact of wavelength of LED light-therapy on endothelial cells. Scientific reports (Nature), Article number: 10700 (2017). It was also shown that exposure of bovine endothelial cells to blue light (wavelength 450 nm, 10 J/cm² from a Waldman lamp) induces a rapid and large reduction in viability followed by the death of the irradiated cells within 24 hours. Sparsa et al., Blue light is phototoxic for B16F10 murine melanoma and bovine endothelial cell lines by direct cytocidal. Anticancer research, 2010, 30.1:143-148. Furthermore, there is evidence that blue light (453 nm) reduces cell proliferation in a dose-dependent manner. Liebman et al., Blue-Light irradiation regulates proliferation and differentiation in Human skin cells. Journal of Investigative Dermatology, 2010, 130:259-269.

Accordingly, the existing studies have suggested that blue light has no effect on endothelial cell migration, proliferation and tubulogenesis, or that blue light decreases their metabolic activity while inducing cell death.

The inventors of the present disclosure have surprisingly discovered that irradiating endothelial cells with blue light under specific conditions has an unexpected technical effect of inducing angiogenesis through increasing the cell viability, the proliferation, the migration, and the tubulogenesis. These effects would be useful, for example, for the healing of chronic wounds, such as during the granulation phase. Accordingly, in some aspects of the invention, a method for inducing angiogenesis is provided that comprises irradiating a target area with visible light, wherein the visible light has a dominant emission wavelength ranging from 435 nm to 520 nm, and effective fluence of the visible light ranges from about 0.15 J/cm² to about 17 J/cm². Dominant wavelength can be defined as the single wavelength that is perceived by the human eye.

In some embodiments, the visible light has an incident power density ranging from about 0.5 mW/cm² to about 20 mW/cm². In some embodiments, the incident power density ranges from about 5 mW/cm² to about 15 mW/cm². In some embodiments, the incident power density ranges from about 8 mW/cm² to about 12 mW/cm². In some embodiments, the power density is about 10 mW/cm²^{.}

In some embodiments, the dominant emission wavelength ranges from 450 nm to 490 nm, or from 450 nm to 460 nm. In some embodiments, the dominant emission wavelength is about 453 nm.

In some embodiments, the irradiation time period ranges from about 5 minutes to about 15 minutes. In some embodiments, the irradiation time period ranges from about 7 minutes to about 15 minutes. The irradiation time period can range from about 10 minutes to about 15 minutes, e.g., it can be about 12 minutes. For example, in some embodiments, the intensity of the light can be from about 8 to about 12 mW/cm² (e.g., about 10 mW/cm²) and the irradiation time period can be from about 10 to about 15 minutes (e.g., about 12 minutes).

In some embodiments, a distance between a target and a source of blue light is between about 1 cm and about 15 cm, or between about 1 cm and about 10 cm, or between about 2 cm and about 7 cm, or about 5 cm.

In some embodiments, the incident power density of blue light is about 10 mW/cm², the irradiation time period is about 12 minutes, and a distance between a target is about 5 cm.

In some embodiments, the irradiation is applied in a continuous mode. In some embodiments, the irradiation is applied in a discontinuous mode.

In some embodiments, a light exposure ratio is from about 5% to about 80%, or from about 10% to about 60%.

In some embodiments, a frequency of the light is from about 0.001 Hz to about 10 Hz.

In some embodiments, effective fluence of the blue light can range from about 0.15 J/cm² to about 17 J/cm², or from about 0.3 J/cm² to about 16 J/cm², or from about 0.3 J/cm² to about 15 J/cm², or from about 0.5 J/cm² to about 15 J/cm², or from about 1 J/cm² to about 15 J/cm², or from about 2 J/cm² to about 15 J/cm², or from about 2 J/cm² to about 10 J/cm², or from about 5 J/cm² to about 10 J/cm². In some embodiments, the effective fluence is from about 5 J/cm² to about 9 J/cm², e.g., about 7.2 J/cm².

Proliferation, migration and tubes formation by endothelial cells is particularly advantageous to enhance the phase of granulation during wound healing. Indeed, it is the key to enhance the wound healing process, as it enables the supply of the wound with oxygen and nutrients, necessary to cell growth.

These unexpected technical effects are achieved through the use of a light emitting device comprising at least one light-emitting diode, or LED for irradiating the cells with blue light.

The unexpected technical effects are achieved through a method for inducing angiogenesis, the method comprising: irradiating a target with blue light, wherein the dominant emission wavelength of blue light ranges from 435 to 520 nm. The term “target,” as used herein, refers to a biological target such as, e.g., a target area of a body, a body region, an organ, or another area of a mammal (a human or animal). The target area can be a mammalian tissue such as, e.g., skin tissue. In some embodiments, the target tissue or cells comprises endothelial cells, such as those in damaged or wounded skin, including skin with a healing impairment, which can result from a genetic disorder (e.g., a genetic disorder that results in chronic blistering of skin or skin lesions) or a result of mechanical/environmental condition (e.g., a pressure sore) or a health condition (e.g., diabetes). In various embodiments, the target tissue is a chronic wound. In some embodiments, the target is ischemic or hypoxic tissue, requiring revascularization or angiogenesis activation. In some embodiments, the target tissue is a systemic scleroderma tissue or atherosclerotic lesions.

U.S. Pat. Application Publication 2019/0175936, which is incorporated herein by reference in its entirety, describes using a visible light with a preferable power density of about 23 mW/cm² to modulate growth and proliferation of dermis cells, in particular of fibroblasts. More specifically, US 2019/0175936 describes that proliferation effects on fibroblasts were observed with an effective fluence of about 6 J/cm² with a power density of about 23 mW/cm² during about 7.5 minutes; and proliferation effects on keratinocytes were observed with an effective fluence of about 1 to 10 J/cm² with a power density of about 23 mW/cm² during about 7.5 minutes. In some embodiments, the invention allows for the growth and proliferation of fibroblasts as well as the induction of angiogenesis, by employing at different times or in alternating fashion, incident power densities and irradiation times that stimulate both fibroblasts as well as endothelial cells. For example, the method may employ a power density of from about 8 mW/cm² to about 12 mW/cm² (e.g., about 10 mW/cm²) to stimulate endothelial cells, alternating in a regimen with a power density of from about 15 mW/cm² to about 20 mW/cm² to stimulate proliferation of fibroblasts.

The described method of promoting or inducing angiogenesis can be used for treatment of various skin conditions, including damaged skin as well as genetic disorders that can result in impaired skin function or healing. Non-limiting examples of the skin conditions are described in PCT/US2015/066147, which is incorporated herein by reference in its entirety, and include blistering diseases of the skin, conditions associated with aging or damaged skin, and immunological disorders involving the skin.

In some embodiments, the target tissue is a wound. For example, the wound can be a chronic wound. In some embodiments, the wound is chronic wound, such as a venous or arterial ulcer, a diabetic ulcer, pressure ulcer (e.g., bedsore), second or third degree burn, skin graft, or amputation wound. Exemplary diabetic ulcers include diabetic leg ulcer or diabetic foot ulcer.

Healing of a wound involves three major chronological processes - the inflammatory phase, the proliferative phase (which includes the phases of granulation and epithelialization), and the phase of remodeling. Certain wounds do not heal properly, and abnormalities of the natural healing process can occur at any of three key stages. Thus, it may be desirable to use a method in accordance with the present disclosure for targeting a tissue that undergoes a certain stage of the healing.

Accordingly, the irradiation can be applied during one or more phases of a tissue healing process. Also, in some embodiments, the irradiation can be administered such that it is targeted in terms of time towards a certain stage, and may not be applied during a certain phase. In some embodiments, different parameters of a visible light can be used for targeting tissue at different stages of the healing process.

Accordingly, in some embodiments, the irradiation is applied during or throughout the granulation or proliferative phase, and optionally a portion of the inflammatory or remodeling phases. In some embodiments, the irradiation is not applied during the inflammatory phase.

In some embodiments, the irradiation is further provided during the remodeling phase. Alternatively, the irradiation is not provided during the remodeling phase.

The irradiation can be applied in various regimens. In some embodiments, the irradiation is provided in a regimen comprising irradiation from about 1 to 5 times per day, to about 1 to about 7 times per week. In some embodiments, the regimen is provided at least during the granulation phase. In some embodiments, the regimen is provided during the granulation phase and at least a portion of the remodeling phase.

The term “wavelength,” as used herein, refers to a distance between two peaks of a wave. The symbol for wavelength is λ (lambda) and it is expressed in nanometers (nm).

The term “dominant emission wavelength,” as used herein, refers to the wavelength or a narrow range of wavelengths of irradiation provided to the target during the majority of the time.

The terms “power density” or light intensity” or “irradiance,” as used herein, refers to the power divided by the area of the target being illuminated and is expressed in mW/cm2. The term “power” refers to the amount of energy transferred per unit time.

The term “effective fluence,” expressed in Joules per square centimeter (J/cm²), is the product of power (mW) and time per spot size (cm²). Effective fluence is the radiant energy received by a surface per unit area, or equivalently the irradiance of a surface, integrated over time of irradiation. The light source device is configured to provide the blue light at a transmitted fluence so that the cell receives the effective fluence. Effective fluence is the product of the incident power density and the irradiation time.

The term “incident power density,” as used herein, refers to the ray light irradiance impacting endothelial cells. It refers to the effective power density received by the cells. One skilled in the art is able to adjust the power density and the distance between the light source and the target to obtain the desired incident power density.

The term “tubulogenesis,” as used herein, refers to the formation of tubes by endothelial cells, i.e. new blood vessels.

The term “irradiation time,” as used herein, refers to the duration of irradiation in continuous mode and to the sum of the irradiation times of each pulse, in discontinuous mode.

As used herein, “about” is defined as ± 10% of the associated numerical value.

One skilled in the art is able to adjust the incident power density and the irradiation time to obtain the desired effective fluence.

The present invention will be described below relative to several specific embodiments. One skilled in the art will appreciate that the present invention may be implemented in a number of different applications and embodiments and is not specifically limited in its application to particular embodiments described herein.

As discussed above, the present disclosure is directed to a method for induction or stimulation of angiogenesis, using blue light irradiation at specific dominant emission wavelength, incident power density, and for a certain irradiation time (also referred to as an irradiation time period).

The invention can be used to provide treatment for many different types of diseases, disorders or conditions. For example, the treatment can be administered, in order to accelerate healing, to subjects having wounds, injuries, or other lesions, including atherosclerotic lesions. The method can also be used to treat a subject having acute or chronic ischemia, including systemic scleroderma, or Raynaud’s disease. Indeed, it was observed by the inventors that monitoring specific conditions of the provided blue light allows increasing cell proliferation and viability, accelerating wound closure and tube formation process.

In some embodiments, specific parameters of the blue light are selected that allow it to induce or promotes angiogenesis, such as a combination of two parameters - a dominant emission wavelength, and an effective fluence. The values of these parameters can be selected such that the blue light administered in accordance with these parameters provides an unexpected effect of increasing proliferation, migration and tubulogenesis of endothelial cells.

In some embodiments, the light is emitted at a wavelength ranging from 435 nm from 520 nm, preferably within a specific dominant emission wavelength of 450-490 nm and preferably within a specific dominant emission wavelength of 450-460 nm. More particularly, the chosen dominant emission wavelength is about 453 nm in some embodiments.

Furthermore, the incident power density of the blue light is selected so as to induce or promote proliferation, migration and tube formation by endothelial cells. To obtain the unexpected effect, the incident irradiance or power density of the blue light is selected to be in ranges from about 0.5 mW/cm² to about 20 mW/cm², or from about 1 mW/cm² to about 15 mW/cm², or from about 5 mW/cm² to about 15 mW/cm², or from about 7 mW/cm² to about 15 mW/cm². In some embodiments, the incident power density of blue light is about 10 mW/cm².

In some embodiments, effective fluence of the blue light can range from about 0.15 J/cm² to about 17 J/cm², or from about 5 J/cm² to about 15 J/cm². In some embodiments, effective fluence of the blue light can range from about 0.15 J/cm² to about 17 J/cm², or from about 0.3 J/cm² to about 16 J/cm², or from about 0.3 J/cm² to about 15 J/cm², or from about 0.5 J/cm² to about 15 J/cm², or from about 1 J/cm² to about 15 J/cm², or from about 2 J/cm² to about 15 J/cm², or from about 2 J/cm² to about 10 J/cm², or from about 5 J/cm² to about 10 J/cm². In some embodiments, the effective fluence is from about 5 J/cm² to about 9 J/cm². In some embodiments, the effective fluence is about 7.2 J/cm².

In some embodiments, the incident power density is from about 8 mW/cm² to about 12 mW/cm² and the irradiation time period is from about 10 minutes to about 15 minutes. For example, in an embodiment, the intensity of the light can be about 10 mW/cm² and the irradiation time period can be about 12 minutes.

In some embodiments, the irradiation time period is selected such that the blue light increases the endothelial cell viability. To obtain the desired effect, the irradiation time period can be from about 5 minutes to about 15 minutes, or from about 7 minutes to about 15 minutes, or from about 10 minutes to about 15 minus. In some embodiments, the irradiation time period is about 12 minutes. In some embodiments, the irradiation time period is about 10 minutes.

In some embodiments, a distance between a target and a source of blue light is between about 1 cm and about 15 cm, or between about 1 cm and about 10 cm, or between about 2 cm and about 7 cm, or about 5 cm.

In some embodiments, the incident power density of blue light is about 10 mW/cm², the irradiation time period is about 12 minutes, and a distance between a target is about 5 cm.

The treatment of endothelial cells may be performed in vitro or in vivo. Indeed, cells may be in culture or may be cells of a tissue of a subject’s body, preferably a mammalian tissue. In some embodiments, the methods described herein are used to facilitate tissue graft attachment and healing, as well as treatment of a donor site.

In embodiments in which the described methods are used for wound healing, treatment can include additional steps of, for example, cleaning the wound bed to facilitate wound healing and closure, including, but not limited to: debridement, sharp debridement (surgical removal of dead or infected tissue from a wound), optionally including chemical debriding agents, such as enzymes, to remove necrotic tissue; wound dressings to provide the wound with a moist, warm environment and to promote tissue repair and healing (e.g., wound dressings comprising hydrogels (e.g., AQUASORB; DUODERM; URGO HYDROGEL), hydrocolloids (e.g., AQUACEL; COMFEEL; ALGOPLAQUE), foams (e.g., LYOFOAM; SPYROSORB; MEPILEX, URGOTUL), and alginates (e.g., ALGISITE; CURASORB; URGOSORB); administration of growth factors to stimulate cell division and proliferation and to promote wound healing e.g. becaplermin; and (iv) soft-tissue wound coverage, a skin graft may be necessary to obtain coverage of clean, non-healing wounds (e.g., autologous skin grafts, cadaveric skin graft, bioengineered skin substitutes (e.g., APLIGRAF; DERMAGRAFT)).

In some embodiments, the described methods can be used in a variety of cosmetic/plastic surgery procedures, including, without limitation, a surgical procedure involving skin grafting and an aesthetic or cosmetic surgery (e.g. a facial plastic surgery procedure including, but not limited to blepharoplasty, rhinoplasty, rhytidectomy, genioplasty, facial implants, otoplasty, hair implantation, cleft lip and cleft palate repair, and/or a body plastic surgery procedure including but not limited to abdominoplasty, brachioplasty, thigh lift, breast reduction, breast augmentation, body contouring, liposuction, hand surgery).

In some embodiments, the application of the blue light having the specific parameters, in accordance with embodiments of the present disclosure, can be performed in combination with use of various therapeutic agents to a targeted tissue.

In some embodiments, a target such as, e.g., endothelial cells may be irradiated either via a continuous exposure or mode or a discontinuous exposure or mode. The “discontinuous exposure” is a type of exposure where the light is sequentially emitted at one or more intervals. For example, the exposure can be interrupted at least twice, at least 5 times, at least 10 times, or at least 15 times. The discontinuous mode can be any cycled or pulsed exposure that should be understood as the sequential emission and disruption of light defined by a specific frequency and a specific period of time. In some embodiments, the continuous and discontinuous modes can be alternated.

A suitable irradiation schedule or regimen can be selected based on various factors, e.g., type of a target to be treated (e.g., wound, burn, skin graft, surgical incision, or another tissue injury or lesion), type of a subject’s condition, subject’s characteristics, previous treatment protocols, etc. A suitable irradiation regimen can be selected based on various factors. For example, in some embodiments, the irradiation is provided in a regimen comprising irradiation from about 1 to 5 times per day, to about 1 to about 7 times per week. This regimen can be used at least during the granulation phase, in the case of wound healing. In some embodiments, the regimen is provided at least during the granulation phase and a portion of the remodeling phase.

In some embodiments, the irradiation is provided in a regimen comprising irradiation once a day, three times a day, five times a day, or ten times a day for at least one week. In some embodiments, the irradiation is provided in a regimen comprising irradiation at least once a day for a week, at least two times a day for a week, at least once a day for two weeks, at least two times a day for two weeks, or at least three times a day for a week. In some embodiments, a course of the treatment can be for more than two weeks - e.g., three, four, or five weeks.

In an embodiment, each sequence of emission/disruption of light can last for a period of time, which can be an attosecond, a femtosecond, a picosecond, a nanosecond, a microsecond, a millisecond, a second, a minute, one hour, or one day. Each sequence can also be defined by its frequency in Hertz, milli-Hertz, micro-Hertz, kilo-Hertz, mega-Hertz, giga-Hertz or tera-Hertz, the frequency being the inverse of the period. According to an embodiment, each sequence is characterized by a frequency of from 0.0001 Hz to 100 Hz, from 0.001 Hz to 100 Hz.

The sequences of emission/disruption may be symmetrical in the sense that a duration of light emission and a duration of light disruption (i.e., a period when the light is not delivered) are the same. Alternatively, the sequences of emission/disruption may be asymmetrical in the sense that the duration of light emission and the duration of light disruption are different.

Each sequence of emission/disruption can be defined by a “light exposure ratio” (or as “duty cycle”), corresponding to the ratio between the duration of light emission and the duration of light disruption. The light exposure ratio is expressed as a percentage and is from 0% to 100%, the ends of the range (0% and 100%) being excluded. A light exposure ratio close to 0% means that the light is disrupted during almost the entire sequence. A light exposure ratio close to 100% means that the light emits during almost the entire sequence. According to an embodiment, the light exposure ratio can be from about 5% to about 80.

In some embodiments, the described methods that make use of blue light are used for enhancement of wound healing. In some embodiments, the method enhances wound healing during the granulation phase. In some embodiments, the method treats ischemic conditions, which can be ischemic conditions in foot ulcer, venous leg ulcers, and pressure ulcers in a diabetic patient.

In embodiments in accordance with the present disclosure, any suitable light source can be used which can be controlled such that its parameters can be adjusted. For example, in some embodiments, the light source can be similar to a light source described in International Application No. PCT/EP2019/05192, which is incorporated herein by reference in its entirety. In some embodiments, the light source can be similar to a light source described in U.S. Pat. Application Publication No. 2019/0175936, which is incorporated herein by reference in its entirety. In some embodiments, a distance between a light emitting element of the light source and a target (e.g., tissue) can be selected and/or adjusted as described in U.S. Pat. Application Publication No. 2019/0175936.

A light source can provide heating. Accordingly, a discontinuous irradiation mode can be used in cases when heat is not desirable. The decision whether to use a continuous irradiation mode or a discontinuous irradiation mode can depend on the exact application and on the total irradiation desired to be delivered. When the discontinuous exposure is preferred, the irradiation time may be determined by the sum of the duration of each light emission.

In some embodiments, the blue light can be delivered directly to a target area. In other embodiments, the blue light irradiation may pass through a product that is in contact with the target area (e.g., skin, or wound or another lesion). For example, in some embodiments, the product that can be in contact with the skin or wound can be a dressing, a strip, a compression element, a Band-Aid©, a patch, a bag, a pouch, a strip, a gel, a film, a film-forming composition, or other product or combination thereof. In some embodiments, the product is a rigid or flexible support, such as, e.g., a dressing. In some embodiments, the dressing comprises at least a hydrocolloid or an adhesive layer that is used to attach the dressing to an intact skin or wound, which would allow blue light to reach the wound at an incident power density sufficient to provide the effects described above. In some embodiments, the rigid or flexible support can be translucent such that it allows at least about 20% of the blue light (having the dominant emission wavelength ranging from 435 nm to 520 nm, or subrange disclosed herein) to pass therethrough, or in some embodiments, at least about 50%, or at least about 70%, or at least about 80%, or at least about 90% of the blue light (having the dominant emission wavelength ranging from 435 to 520 nm, or subrange disclosed herein) to pass therethrough.

### EXAMPLES

### Example 1: Effect of the Blue Light on Endothelial Cell Viability

### Cell Culture

Primary Human Umbilical Vein Endothelial Cells (HUVEC) were isolated from the vein of the umbilical cord and maintained in normal endothelial cell growth medium with 5% FBS and 1% PS in cell culture incubator (37° C., 5% CO2).

### Light Treatment

The plates were irradiated from a distance of 5 cm between the plates and a light source with two incident power densities (10 mW/cm² and 20 mW/cm²) during different exposure of times (7, 12, 15 and 30 minutes). The wavelength used was 453 nm.

### XTT Test (Measurement of the Endothelial Cell Proliferation)

The XTT Cell proliferation test is a method known to the skilled person. The Colorimetric Cell Viability Kit III from PromoCell was used. For the test, 50 µL of labeling-mixture containing labeling reagent and electron coupling reagent was mixed with cell suspension, where the XTT (2,3-bis(2-methoxy-4-nitro-5-sulfophenyl)-2H-tetrazolium-5-carboxanilide) is metabolized to water soluble formazan dye. Only viable cells have the ability to metabolize, hence the formazan is used to directly quantify the proliferation measured by spectrophotometric absorption with Infinite® 200 PRO microplate reader from Tecan Group AG.(Männedorf/Switzerland).

### Results

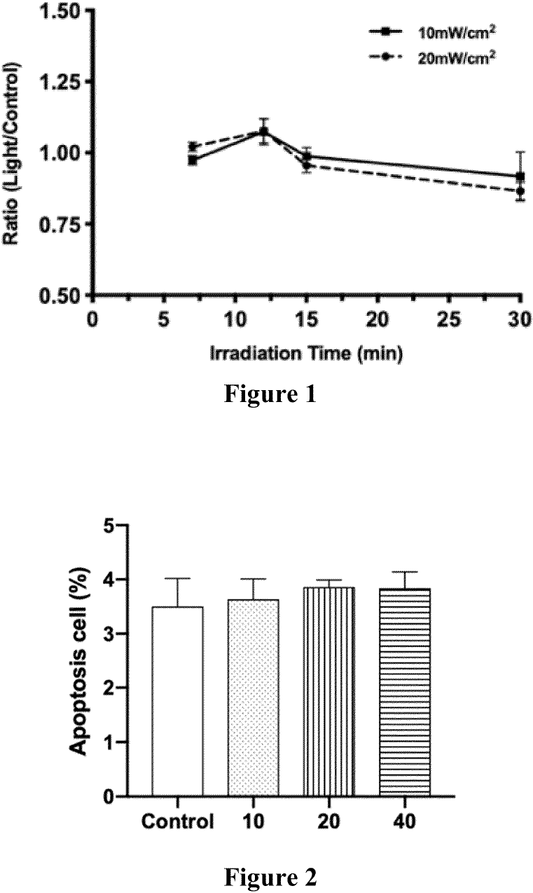

The effect of blue light on endothelial cell viability is illustrated in the graph of FIG. 1. The graph represents the “fold change” in function of the irradiation time. By “fold change”, it is meant the endothelial cell viability with blue light irradiation on the viability of non-irradiated cells (control).

FIG. 1 shows that the exposure of endothelial cells to blue light (453 nm) during a short time of irradiation between 7 minutes and 15 minutes, especially during 12 minutes, increases the endothelial cell viability. On the other hand, the prolonged irradiation time (of more than 15 minutes) decreases the endothelial cell viability. These results show that a short time of irradiation does not decrease the cell viability regardless of the power density, which demonstrates that a short irradiation time with blue light can be used for the treatment of endothelial cells without inducing cytotoxicity.

### Example 2: Effect of Blue Light Irradiation on Cell Apoptosis

### Cell Culture

Primary Human Umbilical Vein Endothelial Cells (HUVEC) were isolated from the vein of the umbilical cord and maintained in normal endothelial cell growth medium with 5% FBS and 1% PS in cell culture incubator (37° C., 5% CO₂).

### Light Treatment

The plates were irradiated from a distance of 5 cm between the plates and a light source with three incident power densities (10 mW/cm², 20 mW/cm², and 40 mW/cm²) during 12 minutes. The wavelength used was 453 nm.

### FACs (Fluorescence Activated Cell Sorting)

The FACs is a method known to the skilled person. For this test, a staining solution of Annexin V-FITC was added to the cell culture. Then, the mixture was incubated for 20 minutes at room temperature in the dark. A binding buffer was added. Immediately after, it was analyzed by flow cytometry.

### Results

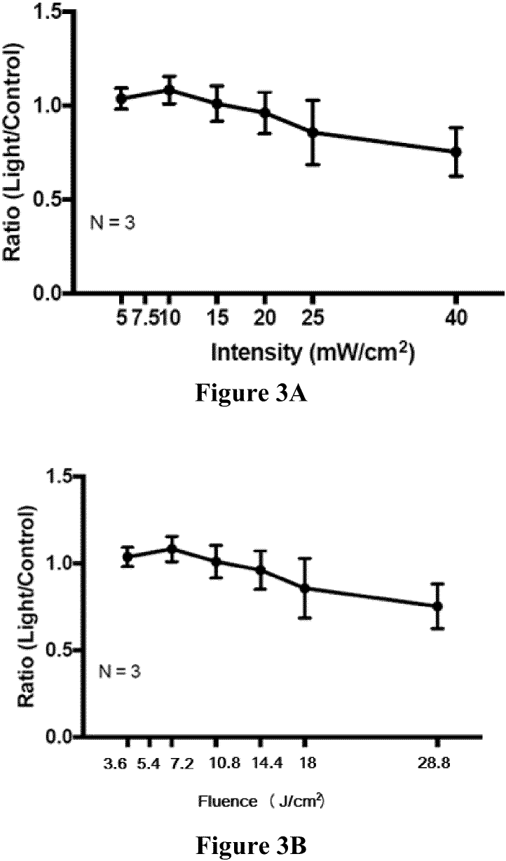

The effect of blue light on endothelial cell apoptosis is illustrated in the graph of FIG. 2. The graph represents the percentage of apoptosis cells on the incident irradiance (control, 10 mW/cm², 20 mW/cm², and 40 mW/cm²).

The graph of FIG. 2 shows that the exposure of endothelial cells to blue light (453 nm) during a short time of irradiation (12 minutes) does not cause cell apoptosis regardless of the used incident irradiance, which indicates that a short irradiation time with blue light (about 12 minutes) can be used for treatment of endothelial cells, for the induction of angiogenesis without any deleterious effect.

### Example 3: Effect of Different Power Densities of Blue Light on Endothelial Cell Proliferation

### Cell Culture

Primary Human Umbilical Vein Endothelial Cells (HUVEC) were isolated from the vein of the umbilical cord and maintained in normal endothelial cell growth medium with 5% FBS and 1% PS in cell culture incubator (37° C., 5% CO₂).

### Light Treatment

The plates were irradiated from a distance of 5 cm between the plates and a light source, with seven different power densities (5 mW/cm², 7.5 mW/cm², 10 mW/cm², 15 mW/cm², 20 mW/cm², 25 mW/cm², and 40 mW/cm²) during 12 minutes, which corresponds to following effective fluences: 3.6 J/cm², 5.4 J/cm², 7.2 J/cm², 10.8 J/cm², 14.4 J/cm², 18 J/cm², and 28.8 J/cm². The wavelength used was 453 nm. The induction of angiogenesis was observed up to about 18 J/cm² and inhibition of angiogenesis was observed at 18 J/cm².

### XTT Test (measurement of the Endothelial Cell Proliferation)

The XTT Cell proliferation test is a known method to the skilled person. The Colorimetric Cell Viability Kit III from PromoCell was used. For the test, 50 µL of labeling-mixture containing labeling reagent and electron coupling reagent was mixed with cell suspension where the XTT (2,3-bis(2-methoxy-4-nitro-5-sulfophenyl)-2H-tetrazolium-5-carboxanilide) is metabolized to water soluble formazan dye. Only viable cells have the ability to metabolize, hence the formazan is used to directly quantify the proliferation measured by spectrophotometric absorption with Infinite® 200 PRO microplate reader from Tecan Group AG.(Mannedorf/Switzerland).

### Results

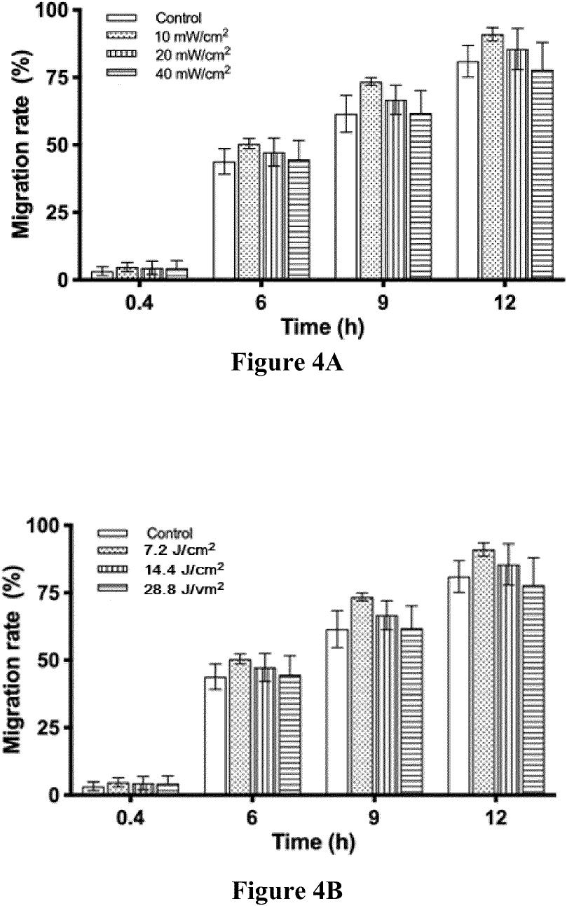

The cell proliferation of endothelial cells is illustrated in the graphs of FIGS. 3A and 3B. The graph represents the “fold change” in function of the irradiation time. By “fold change”, it is meant the endothelial cell proliferation with blue light irradiation on the proliferation of non-irradiated cells (control).

The graphs of FIGS. 3A and 3B show that the exposure of endothelial cells to blue light (453 nm) with an incident power density of between 5 mW/cm² and 15 mW/cm², especially 10 mW/cm², during 12 minutes promotes or induces proliferation of endothelial cells. These results show that effective fluence ranging from 3.6 J/cm² to 10.8 J/cm², particularly 7.2 J/cm², promotes or induces proliferation of endothelial cells.

### Example 4: Effect of Different Power Densities of Blue Light on Endothelial Cells Migration Over Time

### Cell Culture

Primary Human Umbilical Vein Endothelial Cells (HUVEC) were isolated from the vein of the umbilical cord and maintained in normal endothelial cell growth medium with 5% FBS and 1% PS in cell culture incubator (37° C., 5% CO₂).

### Light Treatment

The plates were irradiated from a distance of 5 cm between the plates and a light source with three different power densities (10 mW/cm², 20 mW/cm², and 40 mW/cm²) during 12 minutes, which corresponds to the effective fluence of 7.2 J/ cm², 14.4 J/ cm², and 28.8 J/ cm². The wavelength used was 453 nm.

### Wound Scratch Assay

HUVECs were seeded on 12-well-plate (50,000 cells/well). After 24h, HUVECs have reached 95% confluency. The cell monolayer is scraped in a straight line to create a “scratch” with the wound scratch device. The cell debris was removed by washing the cells twice with 1X PBS which was then replaced with endothelial cell culture medium. HUVECs were treated with blue light irradiation. Images were taken before (0h) and after light irradiation (0, 0.4, 6, 9, and 12 h). The area of the edges of the scratch was analyzed by “ZMFUI” software.

### Results

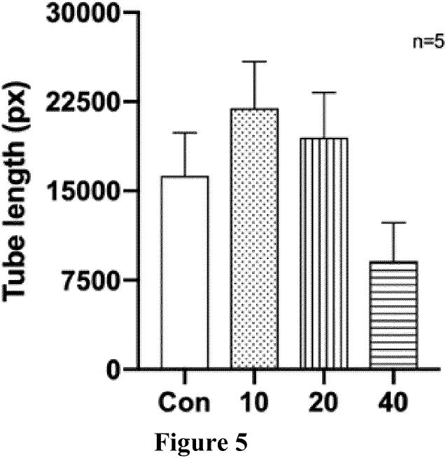

The migration of endothelial cells is illustrated in the graphs of FIGS. 4A and 4B. FIG. 4A shows the endothelial cells migration at different times after irradiation with blue light at three incident power densities (10 mW/cm², 20 mW/cm², and 40 mW/cm²), and for control. FIG. 4B shows the endothelial cells migration at different times after irradiation with blue light at three effective fluences (7.2 J/cm², 14.4 J/cm², and 28.8 J/cm²) and for control. The graphs represent the percentage of cell migration in function of the time after irradiation. FIGS. 4A and 4B show that the exposure of endothelial cells to blue light (at 453 nm) with a low incident power density, especially 10 mW/cm², corresponding to an effective fluence of 7.2 J/ cm², increases cell migration.

### Example 5: Effect of Different Incident Power Densities of Blue Light on Tube Formation by Endothelial Cells

### Cell Culture

Primary Human Umbilical Vein Endothelial Cells (HUVEC) were isolated from the vein of the umbilical cord and maintained in normal endothelial cell growth medium with 5% FBS and 1% PS in cell culture incubator (37° C., 5% CO₂). HUVEC were seeded on a Matrigel® before the light treatment.

### Light Treatment

The plates were irradiated from a distance of 5 cm between the plates and a light source with three different power densities (10 mW/cm², 20 mW/cm², and 40 mW/cm²) during 12 minutes, which corresponds to the effective fluence of 7.2 J/ cm², 14.4 J/ cm² and 28.8 J/ cm². The wavelength used was 453 nm.

### Tube Formation Assay

Matrigel® was distributed evenly on a µ-Slide Angiogenesis (ibidi) and incubated for 30 minutes at 37° C. HUVEC were suspended with serum-free medium and were seeded on the top of the Matrigel® (5000 cells/well). Images were taken 6 hours after irradiation.

### Results

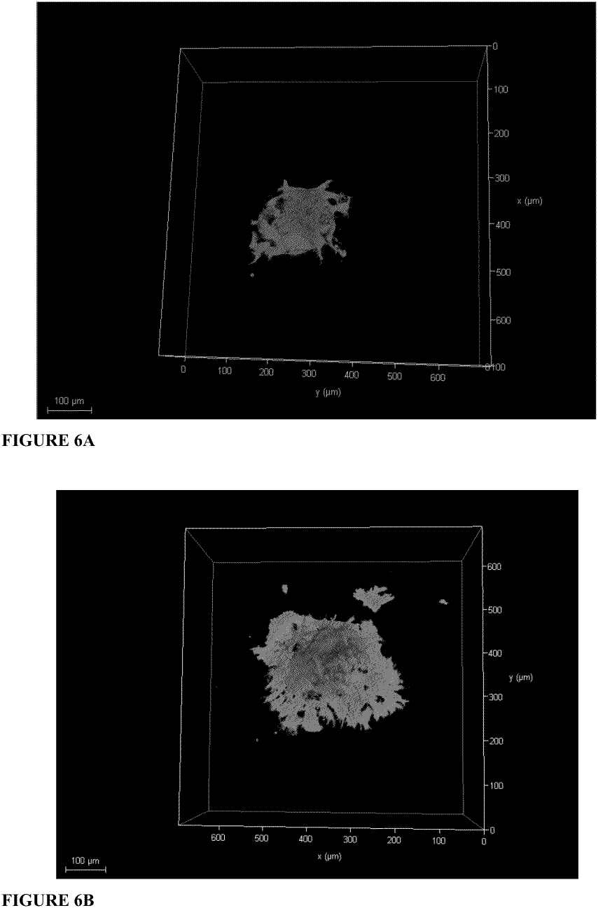

The tube formation is illustrated in the graph of FIG. 5 that represents the tube length in pixels (px) in function of the incident irradiation power density.

FIG. 5 shows that the exposure of endothelial cells to blue light (453 nm) with a low incident power density, particularly 10 mW/cm², corresponding to an effective fluence of 7.2 J/cm² accelerates the process of tube formation.

### Example 6: Assessment of Effect of Different Incident Power Densities on Angiogenesis Using Spheroid Sprouting Assay

### Cell Culture

Primary Human Umbilical Vein Endothelial Cells (HUVEC) were isolated from the vein of the umbilical cord and maintained in normal endothelial cell growth medium with 5% FBS and 1% PS in cell culture incubator (37° C., 5% CO₂). HUVEC spheroids were made by seeding 25 µl drops (400 cells/drop) of cell suspension containing 20% methyl cellulose onto the 20 cm petri-dish. To form spheroids, these drops were incubated upside-down in a cell culture incubator for 24 h. Spheroids were then harvested by spinning down at 200 g for 5 min and embedded in the collagen medium on the µ-Slide.

### Light Treatment

The plates were irradiated from a distance of 5 cm between the plates and a light source with three different power densities (10 mW/cm², 20 mW/cm², and 40 mW/cm²) during 12 minutes, which corresponds to the effective fluence of 7.2 J/ cm², 14.4 J/ cm² and 28.8 J/ cm². The wavelength used was 453 nm.

### Spheroid Sprouting Assay

This assay allows to study angiogenesis in a 3D environment. Endothelial cells are cultured as hanging drops to form spheroids. Those spheroids are embedded into a collagen matrix and tube formation is analyzed 24 h later. Expansion gel was added into each well and incubated in 4° C. overnight. The next day, µ-Slides were moved into 37° C. for 1h and solidified gels were taken out for imaging with Confocal microscope (Leica Microsystems).

### Results

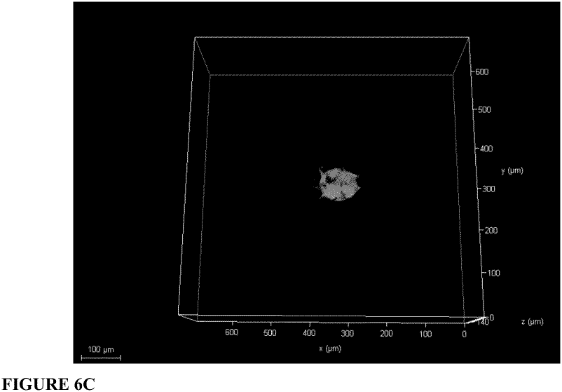

Results of the spheroid sprouting assay are shown in FIGS. 6A, 6B, and 6C. FIG. 6A illustrates an image obtained using control, non-irradiated endothelial cells (HUVEC). FIG. 6B is an image illustrating the results obtained using endothelial cells (HUVEC) irradiated with blue light at 7.2 J/cm² effective fluence, and FIG. 6C is an image illustrating the results using endothelial cells (HUVEC) irradiated with blue light at 28.8 J/cm² effective fluence. Thus, as shown in FIG. 6B, the blue light at 7.2 J/cm² effective fluence causes an increase in the sprout formation as compared to the non-irradiated cells (FIG. 6A), indicating the promotion of angiogenesis. In contrast, the blue light at 28.8 J/cm² effective fluence (FIG. 6C) causes a decrease in the sprout formation as compared to the non-irradiated cells (FIG. 6A), indicating the inhibition of angiogenesis with the increase of the power density.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or illustrative language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

As used herein, all headings are simply for organization and are not intended to limit the disclosure in any manner. The content of any individual section may be equally applicable to all sections.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties. All references, including publications, patent applications, and patents, referenced herein are hereby incorporated by reference to the same extent as if each reference were individually and specifically indicated to be incorporated by reference and were set forth in its entirety herein.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method for inducing angiogenesis, comprising: irradiating a target cell or tissue with visible light for an irradiation time period, wherein the visible light has a dominant emission wavelength ranging from 435 nm to 520 nm, and effective fluence of the visible light ranges from about 0.15 J/cm² to about 17 J/cm2.

2. The method of claim 1, wherein effective fluence of the visible light ranges from about 5 J/cm² to about 15 J/cm2.

3. The method of claim 2, wherein effective fluence of the visible light ranges from about 5 J/cm² to about 10 J/cm².

4. The method of claim 3, wherein the effective fluence is about 7.2 J/cm².

5. The method of claim 1, wherein the visible light has an incident power density ranging from about 0.5 mW/cm² to about 20 mW/cm².

6. The method of claim 6, wherein the visible light has an incident power density ranging from about 5 mW/cm² to about 15 mW/cm².

7. The method of claim 6, wherein the incident power density ranges from about 8 mW/cm² to about 12 mW/cm².

8. The method of claim 7, wherein the incident power density is about 10 mW/cm².

9. The method of any one of claims 1 to 8, wherein the target tissue is skin tissue.

10. The method of any one of claims 1 to 9, wherein the dominant emission wavelength ranges from 450 nm to 490 nm, or from 450 nm to 460 nm.

11. The method of claim 10, wherein the dominant emission wavelength is about 453 nm.

12. The method of anyone of claims 1 to 11, wherein the irradiation time period ranges from about 5 to about 15 minutes.

13. The method of any one of claims 1 to 12, wherein the irradiation time period ranges from about 7 minutes to about 15 minutes.

14. The method of claim 13, wherein the irradiation time period ranges from about 10 minutes to about 15 minutes.

15. The method of claim 14 wherein the irradiation time period is about 12 minutes.

16. The method of any one of claims 1 to 15, wherein the irradiation is applied in a continuous mode.

17. The method of any one of claims 1 to 15, wherein the irradiation is applied in a discontinuous mode.

18. The method of any one of claims 1 to 16, wherein a light exposure ratio is from about 5% to about 80%.

19. The method any one of claims 1 to 18, wherein a frequency of the light is from about 0.001 Hz to about 100 Hz.

20. The method of any one of claims 1 to 19, wherein the target tissue is a wound.

21. The method of claim 20, wherein the target tissue is a chronic wound.

22. The method of claim 20, wherein the wound is venous or arterial ulcer, a diabetic ulcer, a pressure ulcer or sore, a second or third degree burn, a skin graft or donor site, or an amputation wound.

23. The method of any one of claims 1 to 22, wherein the irradiation is applied at least during the granulation phase, and optionally during at least a portion of the inflammatory phase and/or at least a portion of the remodeling phase.

24. The method of claim 23, wherein the irradiation is not applied during the inflammatory phase.

25. The method of claim 23, wherein the irradiation is further provided during the remodeling phase.

26. The method of claim 23, wherein the irradiation is not provided during the remodeling phase.

27. The method of any one of claims 1 to 26, wherein the irradiation is provided in a regimen comprising irradiation from about 1 to 5 times per day, to about 1 to about 7 times per week.

28. The method of claim 27, wherein the regimen is provided at least during the granulation phase.
